(19) United States
(12) Patent Application Publication
(10) Pub. No.: US 2007/0083058 A1
(43) Pub. Date: Apr. 12, 2007

(54) Process for the preparation of aliphatic primary alcohols and related intermediates in such process

(76) Inventors: Georgios Sarakinos, Maastricht (NL); Quirinus Bernardus Broxterman, Munstergeleen (NL)

Correspondence Address: NIXON & VANDERHYE, PC, 901 NORTH GLEBE ROAD, 11TH FLOOR, ARLINGTON, VA, 22203, US

(21) Appl. No.: 10/578,671

(22) PCT Filed: Nov. 17, 2004
(86) PCT No.: PCT/EP04/13160

§ 371 Date: May 9, 2006

(30) Foreign Application Priority Data
Nov. 17, 2003 (EP) 03078564.6

(51) Int. Cl. C07F 7/00; C07C 43/02

(52) U.S. Cl. 556/466; 568/626; 568/671

(57) ABSTRACT

The invention relates to protected alcohol with formula (R¹—O—)ₘPG, wherein R¹ represents a linear, straight-chain alkyl group having 26-30 C-atoms, m is 1 or 2, and PG, forming an ether group in combination with the —O— of the former primary alcohol, represents a protecting group chosen from the group of substituted methyl, substituted ethyl, substituted benzyl and (substituted) silyl groups with at least one substituent on the Si-atom being not a methyl group, in case m=1; and a diol protecting group in case m=2, with the proviso that PG is no saccharide. The invention further relates to process for the preparation of such protected alcohols via an organometallic cross coupling reaction.

### FIELD OF THE INVENTION

High-molecular-weight aliphatic saturated primary alcohols, for instance with 20-40 C-atoms are useful products for use for instance in food or pharmaceutical products. For instance policosanol is a mixture of high-molecular-weight aliphatic primary alcohols with as its main component octacosanal (C28). It is used for instance improvement of serum lipid profiles, which makes it an interesting compound for the prevention and treatment of cardiovascular diseases, and as a cholesterol-lowering additive in foods.

These alcohols, often mixtures thereof, are normally isolated from natural sources, for instance bees wax or plant sources such as sugar cane wax, rice bran wax and birch bark. A disadvantage of these processes is that the isolation is difficult and tedious, and therefore, expensive. Moreover it is difficult—if so desired—to obtain any given compound in pure form from the mixture. Also if a specific mixture of compounds is desired because this is advantageous for the biologic activity, such specific mixture is difficult to obtain.

A synthetic method therefore would be highly desirable. A number of synthetic methods are described in the literature. For instance in WO-A-02/059101 a synthetic route for the preparation of high-molecular-weight linear straight-chain primary alcohols starting from cyclotetradecanone is disclosed. After enamine formation with a cyclic secondary amine, a ring expansion is achieved by reaction with an activated alkanoic acid. The ring is opened in a further transformation and after two more steps the final alcohol is obtained. The synthesis is a 5-step sequence and moreover comprises a.o. a metal hydride reaction which is not attractive on commercial scale from a viewpoint of safety and costs.

In JP 61159591, an electrolytic Kolbe cross-coupling of two different long-chain carboxylic acids is described. An intrinsic element of such cross-coupling is that it leads to a mixture of products. It results in the formation of a 1-alkanoic acid methyl ester that is afterwards reduced to the 1-alkanol. Such processes, however, are commercially less attractive because they require specialized equipment, lead at best to moderate yields and require significant purification procedures.

The present invention now makes it possible to prepare high-molecular-weight aliphatic linear, straight-chain primary alcohols in a simple synthetic process.

Of course, also specific mixtures of high molecular-weight aliphatic linear straight-chain primary alcohols can easily be prepared e.g. by the choice of the starting materials.

Key intermediates in such processes are protected primary alcohols with formula (1)
(R¹—O—)ₘPG (1)

wherein R¹ represents a linear, straight-chain alkyl group having 26-30 C-atoms, m is 1 or 2 and PG, forming an ether group in combination with the —O— of the former primary alcohol, represents a protecting group chosen from the group of substituted methyl, substituted ethyl, substituted benzyl and optionally substituted silyl, groups, with at least one substituent on the Si-atom being not a methyl group, if m=1; or a protecting group for dihydroxy functionalities (diol protecting group) if m=2. The terms substituted methyl, substituted ethyl, substituted benzyl and optionally substituted silyl have the meanings as described by T. W. Greene & PGM. Wuts in Protecting Groups in Organic Synthesis, 3^{rd} Edition, Wiley & Sons; New York, 1999, pp 17-19 and pp 27-148; protecting groups for compounds with dihydroxy functionality are for instance described on pp 201-241 of this same reference (Greene & Wuts). Examples of suitable substituted methyl protective groups are methoxymethyl, methylthiomethyl, benzyloxymethyl, p-methoxytetrahydropyranyl, methoxybenzyloxymethyl, p-nitrobenzyloxymethyl, o-nitrobenzyloxymethyl, guaiacolmethyl, t-butoxymethyl, t-butyldimethylsiloxymethyl, 2-methoxyethoxymethyl, 2,2,2-trichloroethoxymethyl, 2-(trimethylsilyl)ethoxymethyl, methoxymethyl, tetrahydrophyranyl, 1-methoxycyclohexyl, 1,4-dioxan-2-yl and/or tetrahydrofuranyl. Examples of suitable substituted ethyl protecting groups are ethyl, 1-ethoxyethyl, 1-(2-chloroethoxy)ethyl, 1-methyl-1-methoxyethyl, 1-methyl-1-benzyloxyethyl, 1-methyl-1-phenoxyethyl, 2,2,2-trichloroethyl, 2-(benzylthio)ethyl, p-chlorophenyl, t-butyl, allyl and/or propargyl. Examples of suitable substituted benzyl protecting groups are benzyl, p-methoxybenzyl, p-nitrobenzyl, 2,6-dichlorobenzyl, p-phenylbenzyl, 2,6-difluorobenzyl, 2-picolyl, 4-picolyl, p,p′-dinitrobenzhydryl, triphenylmethyl, and/or 1,3-benzodithiolan-2-yl. Suitable substituted silyl protecting groups have sufficient stability under the reaction conditions under which they are formed and/or the work up thereof, of which at least one of the substituents on the Si-atoms is not a methyl group, for example triisopropylsilyl, t-butyldimethylsilyl, t-butyidiphenylsilyl, t-butylmethoxyphenylsilyl triethylsilyl, triisopropylsilyl, dimethylisopropylsilyl, diethylisopropylsilyl, dimethylthexylsilyl, t-butyldimethylsilyl, t-butyldiphenylsilyl, triphenylsilyl, diphenylmethylsilyl, di-t-butylmethylsilyl, t-butoxydiphenylsilyl and/or t-butylmethoxyphenylsilyl. Examples of suitable diol protecting groups are methylene, ethylidene, t-butylmethylidene, 1-t-butylethylidene, 1-phenylethylidene, 1-(4-methoxyphenyl)ethylidene, 2,2,2-trichloroethylidene, isopropyliden, cyclopentylidene, cyclohexylidene, benzylidene, mesitylene, benzophenone, methoxymethylene, ethoxymethylene, di-t-butylsilylene.

Known from WO91/0944 is the use of a mono-, di- or oligosaccharide in the position of PG. However, this type of groups would not be suitable for the process according to the invention, because they carry themselves hydroxyl protecting groups (such as acetyl groups) that interfere (and fall off) during specific coupling conditions described in the present invention (vide infra). Furthermore, the protection step is not suitable to obtain a high yield and requires toxic or expensive reagents (e.g. mercury cyanide, silver oxide, etc) which is not desirable.

Such compounds, and mixtures of such compounds, wherein R¹ represents a linear straight-chain alkyl group with 26-30 C-atoms and PG is as defined above, with the proviso that PG is no benzyl nor a saccharide, are novel intermediates. The invention therefore also relates to such novel intermediates.

In one embodiment the key intermediates with formula (1) are prepared via a so-called organometallic cross-coupling reaction. Such organometallic cross-coupling reactions appeared to work very well, even in the presence of other functional groups.

One example of such an organometallic cross-coupling reaction is schematically as given below.

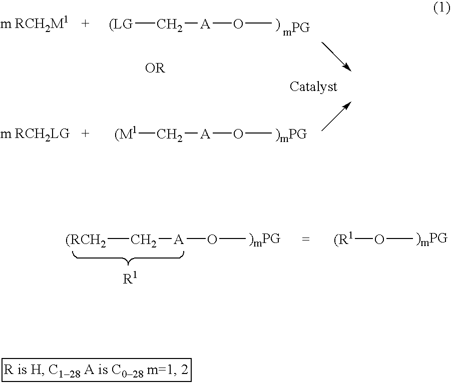

It represents the reaction of a straight-chain nucleophilic organometallic reagent of formula RCH₂M¹ with a linear, straight-chain electrophile of formula (LG-CH₂-A-O—)ₘPG (or a linear, straight-chain electrophile of formula RCH₂LG with a nucleophilic organometallic reagent of formula (M¹-CH₂-A-O—)ₘPG), wherein m=1 or 2, R is H or a linear straight-chain alkyl group with 1-28 C-atoms, M¹ represents Li, Na, K, BZ₂ (wherein Z=OH, an alkyl or alkoxy group, for instance an alkyl or alkoxy group with 1-10 C-atoms, or the 2 Z-groups together may form a 2-7 membered hydrocarbon ring with for instance 2-20 C-atoms, for instance 9-BBN), MgX (wherein X=halogen, for instance Cl, Br, I), ZnX (wherein X=halogen, for instance Cl, Br, I, or CH₂Si(CH₃)₃), MnX (wherein X=halogen, for instance Cl, Br, I), A is a C₀₋₂₈ linear, straight-chain alkylene group, LG represents a leaving group (as, for instance, described in D. S. Kemp & F. Vellaccio, Organic Chemistry, Worth: New York, 1980; pp 99-102, 143-144, 179-180, for example F, Cl, Br, I, OSO₂Ar (Ar represents an aryl group), OMs (OMs represents a mesylate group), OTf (OTf represents a triflate group), OP(O)(OR¹¹)₂ (R¹¹ is an alkyl group, preferably an alkyl group with 1-5 C-atoms), PG is as described above, to produce a linear straight-chain protected alcohol of formula (R¹—O—)ₘPG. The reaction preferably is carried out in the presence of a transition metal catalyst, which may be in the form of a neutral or cationic metal complex ML¹ₐL²_{b}X, an anionic complex Q_{d}[ML¹ₐL²_{b}X_{c}]ₑ, a soluble transition metal nanocluster, or as heterogeneous catalyst wherein the metal in the zero oxidation state is deposited in the form of microcrystalline material on a solid carrier, wherein M can be any transition metal known to catalyze such coupling reactions, for instance Mn, Fe, Cu, Ni or Pd. L¹ and L² are ligands (for instance optionally substituted phosphines and bisphosphines such as triphenylphosphine, bis-diphenylphosphinopropane, 1,1′-diphosphaferrocene (dppf), phosphites or bisphosphites, PN ligands in which there is both a coordinating P atom and a N atom present, N—N ligands such as phenanthrolines), X is an anion which may be a halide, a carboxylate or a composite anion such as BF₄⁻ or PF₆⁻, Q is a cation for instance an alkaline metal ion (for instance sodium, potassium) or a tetraalkylammonium salt, a, b, c, d and e are integers from 0-5. The clusters contain from 2 to many thousands of metal atoms and may carry ligands or anions on the outer rim. Suitable carrier materials for heterogenous catalysts are, for instance, carbon black, silica, aluminum oxide. Particularly when M¹ represents an alkali metal, e.g. Li, Na or K, a metal catalyst is not particularly preferred. Both R and A are saturated (contain no double bonds). In the product of formula (1), R¹ (is RCH₂—CH₂A) is a C₂₆₋₃₀ linear, straight-chain alkyl group and PG is as above. The reaction preferably is performed under an inert atmosphere (e.g. dry nitrogen or dry argon).

In a preferred embodiment of this organometallic coupling, an alkyl magnesium halide, most preferably an alkyl magnesium chloride or bromide (for instance an amount of 1 to 5 equivalents, preferably 1-2 equivalents) is reacted with 1 equivalent of an alkyl halide or alkyl arylsulfonate, alkyl mesylate or alkyl triflate, most preferably with an alkyl fluoride, alkyl chloride, alkyl bromide, alkyl mesylate or alkyl tosylate in the presence of a transition metal catalyst; as for instance described in Terao, J.; Watanabe, H.; Ikumi, A.; Kuniyasu, H.; Kambe, N. J. Am. Chem. Soc. 2002, 124, 4222-4223, and Terao, J.; Ikumi, A., Kuniyasu, H; Kambe, N. J. Am. Chem. Soc. 2003, 125, 5646-5647. Preferably the reaction is carried out in the presence of a solvent. Suitable solvents are for instance ethyl ether, tetrahydrofuran (THF), i-propyl ether di-n-propyl ether, dimethoxyethane (DME) or methyl t-butyl ether or mixtures of these solvents with a dipolar aprotic solvent such as NMP, DMF or DMA (dimethylacetamide) in any proportion, most preferably THF, and the concentration of each of the reactants is preferably between 0.2 and 3 molar. The transition metal catalyst is based on a transition metal M chosen preferably from Mn, Fe, Cu, Ni, Pd. They can be in the form of pre-formed complexes or made in situ from a catalyst precursor and one or more ligands. If desired an activator (for instance a base, such as an alkoxide, or a reducing agent, such as NaBH₄)⁻ may be added to these complexes. Suitable sources of catalyst precursors are for instance precursors of Cu^{I} (for example CuCl, CuI, CuOTf), Cu^{II} (for example CuCl₂, Li₂CuCl₄), Ni⁰ (for example Ni(COD)₂), Ni^{II} (for example NiCl₂, Ni(acac)₂, NiBr₂), or Pd^{II} (for example PdCl₂, Pd(OAc)₂, Pd₂(dba)₃), Mn^{III} (for example MnCl₃, Mn(acac)₃) or Fe^{III} (for example Fe(acac)₃). Preformed catalysts can also be used, for example (PPh₃)₂NiCl₂, (dppp)NiCl₂ or (dppf)NiCl₂. The amount of catalyst that is used is calculated with respect to the electrophile and is preferably lower than 0.05 equivalents, more preferably between 0.001 and 0.03 equivalents calculated with respect to the electrophile. Preferably less than 4 equivalents of each ligand with respect to the amount of metal M are used. Optionally, the reaction is run in the presence of a 1,3-diene, for example 1,3-butadiene, isoprene or 2,3-dimethyl-1,3-butadiene, in a relative amount of 0.1 to 2.0 equivalents calculated with respect to the electrophile. The temperature at which the reaction is performed preferably lies between −78 to 80° C., more preferably between −20 and 80° C. The reaction time required is preferably between 1 and 24 hours.

In a second preferred embodiment, the nucleophilic reagent may be of the general structure RCH₂ZnX (wherein for example X=Br, I or CH₂SiMe₃, and R is as above); as for instance described in Jensen, A. E.; Knochel, P. J. Org. Chem. 2002, 67, 79-85. Preferably, an alkylzinc iodide (preferred amount 1.05-1.5 equivalents calculated with respect to the electrophile) is reacted with 1 equivalent of an alkyl bromide or iodide, preferably iodide, optionally in the presence of a tetraalkylammonium halide R³₄NX, wherein each R³, independently, represents an alkyl group, for instance an alkyl group with 1-16 C-atoms and X represents a halogen, for instance Cl, Br or I, for instance n-Pr₄NI, n-Bu₄NBr, n-Bu₄NI (preferred amount 1-5 equivalents with respect to the alkyl halide), and optionally in the presence of a styrene preferably a mono- or polyfluorinated styrene, such as m-fluorostyrene or p-fluorostyrene (preferred amount 0.05-0.30 equivalents calculated with respect to the electrophile) and a Nil catalyst, such as NiCl₂, Ni(acac)₂, NiBr₂, (PPh₃)₂NiCl₂, (dppp)NiCl₂, in a relative amount between 0.01 and 0.20 equivalents calculated with respect to the electrophile. The reaction preferably is carried out in the presence of a solvent. Suitable solvents that may be used are for instance ethers, NMP, DMF or mixtures thereof. The reaction preferably is run at temperatures between −30 and 25° C. The reaction time required preferably is between 2 and 30 h.

In a third preferred embodiment, the nucleophilic reagent may be of the general structure RCH₂BR⁴₂ (wherein each R⁴ independently represents an alkyl group, for instance an alkyl group with 1-10 C-atoms, or may be part of a ring, for instance as in 9-BBN), RCH₂B(OH)₂ or RCH₂B(OR⁴)₂, wherein R is as above, as for instance described in Netherton, M. R.; Dai, C.; Neuschütz, K.; Fu, G. C. J. Am. Chem. Soc. 2001, 123, 10099-10100, Kirchhoff, J. H.; Dai, C.; Fu, G. C. Angew. Chem. Int. Ed. 2002, 41, 1945-1947, Kirchhoff, J. H.; Netherton, M. R.; Hills, I. D.; Fu, G. C. J. Am. Chem. Soc. 2002, 124, 13662-13663, and Netherton, M. R.; Fu, G. C. Angew. Chem. Int. Ed. 2002, 41, 3910-3912.

In one embodiment an alkyl-(9-BBN) reagent (preferred amount 1-3 equivalents, calculated with respect to the amount of electrophile), is reacted with for instance an alkyl chloride, bromide or tosylate, preferably a bromide or a tosylate. The reaction is catalyzed by a source of Pd⁰ or Pd^{II}, such as Pd(OAc)₂, PdCl₂, or Pd₂(dba)₃, preferably Pd(OAc)₂, in an amount calculated with respect to the electrophile of 0.01-0.10 equivalents. Addition of a stabilizing ligand for the metal may be beneficial. Suitable examples of such stabilizing ligands are PR⁵₃ (wherein each R⁵ independently represents a, for instance C1-C20, alkyl, aryl, heteroaryl, etc. group, e.g. P(i-Pr)₃, P(t-Bu)₃, PCy₃ (Cy=cyclohexyl), PPh₃, P(2-furyl)₃, P(t-Bu)₂Me), preferably PCy₃. The source of the phosphine ligand may also be the corresponding phosphonium salt (less susceptible to oxidation), such as (HP(t-Bu)₂Me)BF₄. The relative amount of the phosphine may be 0.05-0.20 equivalents calculated with respect to the electrophile, preferably in a molar ratio 2:1 to Pd. In addition, as a rule a base is added, for instance a phosphate salt such as Na₃PO₄.H₂O or K₃PO₄.H₂O; an alkali metal hydroxide, for instance NaOH, KOH, LiOH or CsOH; or a bulky alkoxide base such as LiOt-Bu, NaOt-Bu or KOt-Bu, in a proportion of 1-4 equivalents calculated with respect to the electrophile. The reaction preferably is carried out in the presence of a solvent. Suitable solvents that can be used are the ethers mentioned above, also dioxane or a bulky alcohol, such as t-amyl alcohol. THF is preferably used as the solvent with alkyl-(9-BBN) derivatives and t-amyl alcohol with alkyl boronic acids. In some cases, the addition of one or two equivalents of water with respect to the electrophile may be beneficial. The reaction preferably is run at temperatures between 25 and 100° C. (higher temperatures are preferred for more unreactive alkyl chloride electrophiles).

In another embodiment, the nucleophilic reagent may be of the general structure RCH₂M¹ with M¹=Li, Na, K and R is as above. It is reacted preferably with an alkyl halide or tosylate, preferably an alkyl bromide, iodide or tosylate. A metal catalyst is not particularly preferred in these cases. The stoichiometries of these reactions are as above (for instance an excess organometallic reagent, preferably 1-3 equivalents, most preferably 1-1.5 equivalents). The preferred solvents are here the ethers mentioned above (preferably THF), but also toluene can be suitably used, especially when higher reaction temperatures are required.

Subsequently the protected alcohols with formula (1) and mixtures thereof can be converted into the desired, corresponding unprotected alcohols with formula R¹OH and mixtures thereof wherein R¹ is as defined above.

Processes for deprotection are commonly known in the art. The skilled person can easily find a suitable method for their case. Deprotection can be depicted schematically as follows:

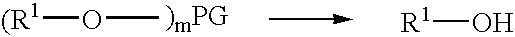

Some examples of deprotection reactions are given below.

### EXAMPLE 1

### Removal of a Methoxymethyl Group

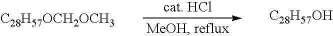

An example of a removal of a common PG from a protected higher (C28) alkanol is shown above. The PG methoxymethyl ether can for instance be cleaved under acidic conditions in methanol, at reflux.

### EXAMPLE 2

### Removal of a Benzyl Group

Another PG, for example, a benzyl group, can be removed under reductive conditions, in the presence of hydrogen gas and a palladium catalyst:

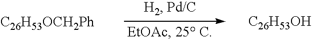

### EXAMPLE 3

### Removal of a t-Butyldimethylsilyl Group

In yet another example, where the PG is a t-butyldimethylsilyl group, deprotection can be easily achieved, for instance, by fluoride ion in THF at 25° C., originating from, for example, tetrabutylammonium fluoride:

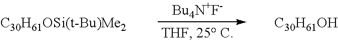

For further details about the above and other protecting groups, see T. W. Greene & P. G. M. Wuts in Protecting Groups in Organic Synthesis, 3^{rd} Edition, Wiley & Sons: New York, 1999; pp 27-148.

### EXAMPLE 4

### Synthesis and Deprotection Reactions

The examples as depicted in the following schematic representation were conducted and are described below as examples I-IX.

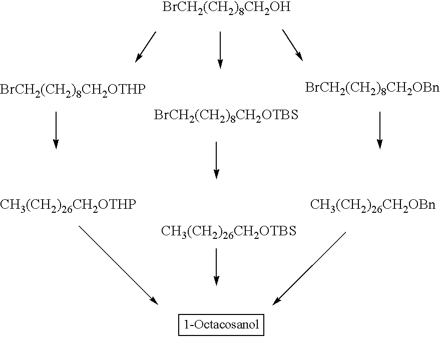

### EXAMPLE I

### Preparation of a Protected Electophiles

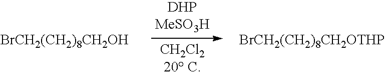

2-(10-Bromo-decyloxy)-tetrahydro-pyran. 10-bromo-decan-1-ol was prepared according to J. Org Chem. 2000, 65, 5837-5838 by J. Michael Chong, et al. To a stirred solution of 10-bromo-decan-1-ol (3.79 g, 16.0 mmol) and 3,4-Dihydro-2H-pyran (2.03 g, 2.20 mL, 24.1 mmol) in CH₂Cl₂ (50 mL) at 20° C., MeSO₃H (50 μL, 74 mg, 0.771 mmol) was added and the mixture was stirred for 3 h. Aq. sat. NaHCO₃ (50 mL) was then added, the phases were shaken vigorously and then separated. The organic phase was concentrated in vacuo (20 mbar, 50° C.) and the crude liquid product was purified by a short silica gel flash chromatography using 1:99 & 1:49 MTBE:petroleum benzene as eluent. The product (3.73 g, 11.6 mmol; 72% yield based on 10-bromo-decan-1-ol) was obtained as a colorless liquid.

Reaction conditions were not optimized.

### EXAMPLE II

### Preparation of a Protected Electrophile

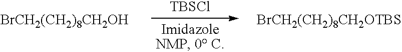

(10-Bromo-decyloxy)-t-butyl-dimethyl-silane. To a stirred solution of 10-bromo-decan-1-ol (2.44 g, 10.3 mmol) in NMP (13 mL) at 0° C., TBSCl (1.66 g, 11.0 mmol) was added followed by imidazole (0.716 g, 10.5 mmol) portion wise (3×0.200 g & 1×0.116 g) in 15 min intervals. After the last portion of imidazole had been added, the reaction was stirred for an additional 2 h at 0° C. and then it was poured into water (100 mL). The product was extracted into pentane (100 mL), the organic phase was concentrated in vacuo (20 mbar, 50° C.) and the crude liquid was purified by filtration through a short silica gel column, using 1:9 MTBE:petroleum benzene as eluent. The product was obtained as a colorless liquid (3.12 g, 8.88 mmol, 86% yield based on 10-bromo-decan-1-ol).

Reaction conditions were not optimized.

### EXAMPLE III

### Preparation of a Protected Electrophile

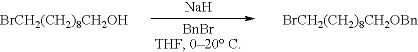 (10-Bromo-decyloxymethyl)-benzene. NaH (60% oil disp, 0.83 g, 20.7 mmol) was suspended in dry THF (60 mL) and the mixture was cooled to 0° C. and benzyl bromide (3.08 g, 2.14 mL, 18.0 mmol) was added, followed by a dropwise addition of 10-bromo-decan-1-ol (4.57 g, 19.3 mmol). 15 min later, the cold bath was removed and the reaction was stirred at 20° C. for 60 h, and then poured slowly into cold, aq. sat. NaHCO₃ (75 mL). The mixture was allowed to warm to 20° C. and extracted with petroleum benzene (120 mL+50 mL). The combined organic layers were concentrated in vacuo (20 mbar, 50° C.) and the crude liquid was purified by silica gel flash chromatography (100:0 to 19:1 petroleum benzene:MTBE as eluent) to give the product as a colorless liquid (3.83 g, 11.7 mmol, 60% yield based on 10-bromo-decan-1-ol).

Reaction conditions were not optimized.

### EXAMPLE IV

### Organometallic Coupling

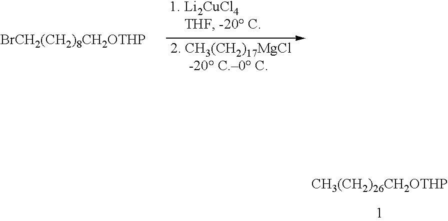

2-Octacosyloxy-tetrahydro-pyran 1. To a stirred solution of 2-(10-Bromo-decyloxy)-tetrahydro-pyran (2.08 mmol) in THF (1.5 mL) at −20° C. under a nitrogen atmosphere, a solution of Li₂CuCl₄ (0.1 M in THF, 1.46 mL, 0.146 mmol) was added over a period of 5-10 min, keeping the temperature at −20° C. The bright yellow solution was stirred for 15 min at −20° C. and then octadecylmagnesium chloride (0.5 M in THF, 9.18 mL, 4.56 mmol) was added during a period of 10 min, while maintaining the temperature at −20° C. The resulting brownish, heterogeneous mixture was allowed to warm up slowly over a period of 75 min to 0° C. and was then quenched with aq. sat. NH₄Cl (50 mL). The products were extracted into a 1:1 mixture of MTBE and petroleum benzene (100 mL). The organic phase was separated and the solvents were evaporated in vacuo (20 mbar, 50° C.). The residual waxy product was purified by silica gel flash column chromatography using 1:99 and 1:49 MTBE:petroleum benzene as eluent. The first fractions contained the C18 hydrocarbon by-product (discarded) and the following ones, containing the desired product, were pooled. After removal of the solvents in vacuo (20 mbar, 50° C.) the product was obtained as colorless oil [765 mg, 1.55 mmol, 74% yield based on 2-(10-Bromo-decyloxy)-tetrahydro-pyran], which solidified to a wax upon cooling to r.t. ¹H NMR analysis indicated that the purity of the product was >95%.

Reaction conditions were not optimized.

### EXAMPLE V

### Organometallic Coupling

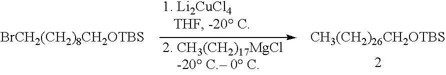

t-Butyl-dimethyl-octacosyloxy-silane 2. Same procedure as for 1. The yield after chromatographic purification was 60% (575 mg, 1.10 mmol). ¹H NMR analysis indicated that the purity of the product was >95%.

Reaction conditions were not optimized.

### EXAMPLE VI

### Organometallic Coupling

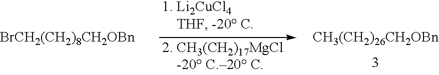

Benzyl 1-octacosanol 3. Same procedure as for 1. The yield after chromatographic purification was 70% (397 mg, 0.79 mmol). ¹H NMR analysis indicated that the purity of the product was >95%.

Reaction conditions were not optimized.

### EXAMPLE VII

### Deprotection Reaction

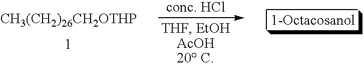

2-Octacosyloxy-tetrahydro-pyran 1 (765 mg, 1.55 mmol) was dissolved in THF (8 mL), 95% EtOH (1 mL) and acetic acid (1 mL). To the homogeneous solution, aq. HCl (0.20 mL, 2.0 M, 0.40 mmol). The reaction was stirred at 20° C. for 16 h, during which time solid appeared. Petroleum benzene was added (30 mL), the mixture was further stirred for a few minutes and the solid product was collected on a fritted funnel, under suction, washed with MeOH (20 mL) and more petroleum benzene (20 mL) and allowed to air-dry. 1-Octacosanol was obtained as a colorless solid (510 mg, 1.24 mmol, 80% yield).

Reaction conditions were not optimized.

### EXAMPLE VIII

### Deprotection Reaction

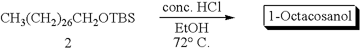

t-Butyl-dimethyl-octacosyloxy-silane 2 (500 mg, 0.952 mmol) was suspended in abs. EtOH (12 mL) and the mixture was heated to 72° C. To the homogeneous solution, aq. HCl (0.20 mL, 12.0 M, 2.40 mmol) was added. The reaction was stirred at 72° C. for 5 h, then most of the solvent was evaporated in vacuo (20 mbar, 60° C.) and to the residue, MeOH (20 mL) was added, the mixture was stirred for a few minutes and then 1-octacosanol was isolated on a fritted funnel as above (300 mg, 0.730 mmol, 77% yield).

Reaction conditions were not optimized.

### EXAMPLE IX

### Deprotection Reaction

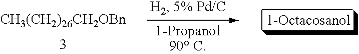

Benzyl 1-octacosanol 3 (375 mg, 0.749 mmol) and 5% Pd/C (36.3 mg, Johnson Matthey) were suspended in 1-Propanol (6 mL) and with good stirring the mixture was heated to 90° C. under a H₂ pressure of 5 bar for 18 h in an Endeavor apparatus. The reaction mixture was then allowed to cool to 20° C. The solidified solution was diluted with THF (5 mL) and re-dissolved with heating and the catalyst was filtered off through a short plug of decalite. The THF was then removed in vacuo (20 mbar, 60° C.) and MeOH (20 mL) was added and the mixture was stirred at 20° C. for 10 min. 1-Octacosanol was recovered as above on a fritted funnel. (250 mg, 0.608 mmol, 81% yield).

Reaction conditions were not optimized.

1. Protected alcohol with formula (1)
(R¹—O—)ₘPG (1)
wherein R¹ represents a linear, straight-chain alkyl group having 26-30 C-atoms, m is 1 or 2, and PG represents a protecting group chosen from the group of substituted methyl ethers, substituted ethyl ethers, substituted benzyl ethers and (substituted) silyl ethers with at least one substituent on the Si-atom being not a methyl group, in case m=1; and a diol protecting group in case m=2, with the proviso that PG is no saccharide.

2. Process for the preparation of a protected alcohol according formula (1)
(R¹—O—)ₘPG (1)
wherein R¹ represents a linear, straight-chain alkyl group having 26-30 C-atoms, m is 1 or 2, and PG represents a protecting group chosen from the group of substituted methyl ethers, (substituted) ethyl ethers, (substituted) benzyl ethers and (substituted) silyl ethers with at least one substituent on the Si-atom being not a methyl group, in case m=1; and a diol protecting group in case m=2, with the proviso that PG is no saccharide, via an organometallic cross coupling reaction wherein a linear, straight-chain nucleophilic organometallic reagent of formula RCH₂M₁ is reacted with a linear, straight-chain electrophile of formula LG-CH₂-A-O—)ₘPG (or a linear, straight-chain electrophile of formula RCH₂-LG with a nucleophilic organometallic reagent of formula (M₁CH₂-A-O—)ₘPG), wherein R is H or a linear, straight-chain alkyl group with 1-28 C-atoms, M1 represents Li, Na, K, BZ₂, wherein each Z independently represents OH, an alkyl group or an alkoxy group, or the 2 Z-groups together form a hydrocarbon ring, MgX, wherein X=halogen, ZnX, wherein X=halogen or CH₂Si(CH₃)₃), or MnX, wherein X=halogen, A is a C₀₋₂₈ linear, straight-chain alkylene group, LG represents a leaving group, and m and PG are as described above.

3. Process according to claim 2, wherein the organometallic cross coupling reaction is performed in the presence of a transition metal catalyst and wherein M¹ represents MgX with X is halogen.

4. Process according to claim 3, wherein the nucleophilic organometallic reagent reacts with an alkyl halide, alkyl arylsulfonate or alkyl mesylate.

5. Process according to claim 2, wherein first the protected alcohol with formula (1) is prepared according to claim 2 and subsequently the protected alcohol is subjected to deprotection.
